(19) United States
(12) Patent Application Publication
(10) Pub. No.: US 2003/0019763 A1
(43) Pub. Date: Jan. 30, 2003

(54) Apparatus and method for separation of biological contaminants

(76) Inventors: Brendon Conlan, Sydney (AU); Tracey Ann Edgell, Dee Why (AU); May Lazar, Wattle Grove (AU); Chenicheri Hariharan Nair, Old Greenwich, CT (US); Elizabeth Jean Seabrook, Lane Cove (AU); Thomas Norman Turton, Sydney (AU)

Correspondence Address: James D. Jacobs, Esq., Baker & McKenzie, 805 Third Avenue, New York, NY, 10022, US

(21) Appl. No.: 10/037,004

(22) Filed: Jan. 2, 2002

Related U.S. Application Data
(63) Continuation-in-part of application No. 09/877,371, filed on Jun. 8, 2001.

(30) Foreign Application Priority Data
Dec. 23, 1999 (AU) PP7906

(51) Int. Cl. C25F 1/00; C25C 7/00; C25B 9/00; C25D 17/00

(52) U.S. Cl. 205/688; 204/252

(57) ABSTRACT

A method of removing a biological contaminant from a mixture containing a biomolecule and the biological contaminant, the method comprising: (a) placing the biomolecule and contaminant mixture in a first solvent stream, the first solvent stream being separated from a second solvent stream by an electrophoretic membrane; (b) selecting a buffer for the first solvent stream having a required pH; (c) applying an electric potential between the two solvent streams causing movement of the biomolecule through the membrane into the second solvent stream while the biological contaminant is substantially retained in the first sample stream, or if entering the membrane, being substantially prevented from entering the second solvent stream; (d) optionally, periodically stopping and reversing the electric potential to cause movement of any biological contaminants having entered the membrane to move back into the first solvent stream, wherein substantially not causing any biomolecules that have entered the second solvent stream to re-enter first solvent stream; and (e) maintaining step (c), and optional step (d) if used, until the second solvent stream contains the desired purity of biomolecule.

This application is a continuation-in-part of application Ser. No. 09/877,371, filed Jun. 22, 2001.

### TECHNICAL FIELD

The present invention relates to methods for the removal of biological contaminants, particularly removal of biological contaminants from biological preparations.

### BACKGROUND ART

The modem biotechnology industry is faced with a number of problems especially concerning the processing of complex biological solutions which ordinarily include proteins, nucleic acid molecules and complex sugars and which are contaminated with unwanted biological materials. Contaminants include microorganisms such as bacteria and viruses or biomolecules derived from microorganisms or the processing procedure. The demand is, therefore, for a high purity, scalable separation, which can be confidently used both in product development and production, which in one step will both purify macromolecules and separate these biological contaminants.

Viruses are some of the smallest non-cellular organisms known. These simple parasites are composed of nucleic acid and a protein coat. Viruses are typically very small and range in size from 1.5×10⁻⁸ m to 5.0×10⁻⁵ m. Viruses depend on the host cells that they infect to reproduce by inserting their genetic material into the host. Often literally taking over the host's function. An infected cell produces more viral protein and genetic material, often instead of its usual products. Some viruses may remain dormant inside host cells. However, when a dormant virus is stimulated, it can enter the lytic phase where new viruses are formed. Self-assemble occurs and burst out of the host cell results in killing the cell and releasing new viruses to infect other cells. Viruses cause a number of diseases in humans including smallpox, the common cold, chicken pox, influenza, shingles, herpes, polio, rabies. Ebola, hanta fever, and AIDS. Some types of cancer have been linked to viruses.

Pyrogens are agents which induce fever. Bacteria are a common source for the production of endotoxins which are pyrogenic agents. Furthermore, another detrimental effect of endotoxins is their known adjuvant effect which could potentially intensify immune responses against therapeutic drugs. The endotoxin limit set by the Food and Drug Administration (FDA) guidelines for most pharmaceutical products is for a single dose 0.5 ng endotoxin per kilogram body weight or 25 ng endotoxin/dose for a 50 kg adult. Due to their size and charge heterogeneity, separation of endotoxins from proteins in solution can often be difficult. Endotoxin inactivation by chemical methods are unsuitable because they are stable under extremes of temperature and pH which would destroy the proteins. Furthermore, due to their amphipathic nature, endotoxins tend to adhere to proteins in a fashion similar to detergents. In such cases, endotoxin activity often clusters with the protein when chromatographic procedures such as ion exchange chromatography or gel filtration are employed.

Presently, the purification of biomolecules is sometimes a long and cumbersome process especially when purifying blood proteins. The process is made all the more complex by the additional step of ensuring the product is “bug” free. The costs associated with this task is large and further escalates the purification costs in total. The Gradiflow technology rapidly purifies target proteins with high yield. For example, a proteins like fibrinogen (a clotting protein) can be separated in three hours using the Gradiflow while the present industrial separation is 3 days. Certain monoclonal antibodies can be purified in 35 minutes compared to present industrial methods which take 35 hours.

The membrane configuration in the Gradiflow enables the system to be configured so that the purification procedure can also include the separation of bacteria viruses and vectors. It has now been found by the present inventors that appropriate membranes can be used and the cartridge housing the membrane configured to include separate chambers for the isolated bacteria and viruses.

The Gradiflow Technology

Gradiflow is a unique preparative electrophoresis technology for macromolecule separation which utilizes tangential flow across a polyacrylamide membrane when a charge is applied across the membrane (AU 601040). The general design of the Gradiflow system facilitates the purification of proteins and other macromolecules under near native conditions. This results in higher yields and excellent recovery.

In essence the Gradiflow technology is bundled into a cartridge comprising of three membranes housed in a system of specially engineered grids and gaskets which allow separation of macromolecules by charge and/or molecular weight. The system can also concentrate and desalt/dialyse at the same time. The multimodal nature of the system allows this technology to be used in a number of other areas especially in the production of biological components for medical use. The structure of the membranes may be configured so that bacteria and viruses can be separated at the point of separation—a task which is not currently available in the biotechnology industry and adds to the cost of production through time delays and also because of the complexity of the task.

### DISCLOSURE OF INVENTION

In a first aspect, the present invention consists in a method of removing a biological contaminant from a mixture containing a biomolecule and the biological contaminant, the method comprising:

(a) placing the biomolecule and contaminant mixture in a first solvent stream, the first solvent stream being separated from a second solvent stream by an electrophoretic membrane;

(b) selecting a buffer for the first solvent stream having a required pH;

(c) applying an electric potential between the two solvent streams causing movement of the biomolecule through the membrane into the second solvent stream while the biological contaminant is substantially retained in the first sample stream, or if entering the membrane, being substantially prevented from entering the second solvent stream;

(d) optionally, periodically stopping and reversing the electric potential to cause movement of any biological contaminants having entered the membrane to move back into the first solvent stream. wherein substantially not causing any biomolecules that have entered the second solvent stream to re-enter first solvent stream: and

(e) maintaining step (c), and optional step (d) if used, until the second solvent stream contains the desired purity of biomolecule.

In a second aspect. the present invention consists in a method of removing a biological contaminant from a mixture containing a biomolecule and the biological contaminant, the method comprising:

(a) placing the biomolecule and contaminant mixture in a first solvent stream, the first solvent stream being separated from a second solvent stream by an electrophoretic membrane;

(b) selecting a buffer for the first solvent stream having a required pH;

(c) applying an electric potential between the two solvent streams causing movement of the biological contaminant through the membrane into the second solvent stream while the biomolecule is substantially retained in the first sample stream, or if entering the membrane, being substantially prevented from entering the second solvent stream;

(d) optionally, periodically stopping and reversing the electric potential to cause movement of any biomolecule having entered the membrane to move back into the first solvent stream, wherein substantially not causing any biological contaminants that have entered the second solvent stream to reenter first solvent stream; and

(e) maintaining step (c), and optional step (d) if used, until the first solvent stream contains the desired purity of biomolecule.

In the first and second aspects of the present invention, preferably the biomolecule is selected from the group consisting of blood protein, immunoglobulin, and recombinant protein.

The biological contaminant can be a virus, bacterium, prion or an unwanted biomolecule such as lipopolysaccharide, toxin or endotoxin.

Preferably, the biological contaminant is collected or removed from the first stream.

Preferably, the buffer for the first solvent stream has a pH lower than the isoelectric point of biomolecule to be separated.

In a further preferred embodiment of the first aspect of the present invention, the electrophoretic membrane has a molecular mass cut-off close to the apparent molecular mass of biomolecule. It will be appreciated. however, that the membrane may have any required molecular mass cut-off depending on the application. Usually, the electrophoretic membrane has a molecular mass cut-off of between about 3 and 1000 kDa. A number of different membranes may also be used in a desired or useful configuration.

The electric potential applied during the method is selected to ensure the required movement of the biomolecule, or contaminant if appropriate, through the membrane. An electric potential of up to about 300 volts has been found to be suitable. It will be appreciated, however, that greater or lower voltages may be used.

The benefits of the method according to the first aspect of the present invention are the possibility of scale-up, and the removal of biological contaminants present in the starting material without adversely altering the properties of the purified biomolecule.

In a third aspect, the present invention consists in use of Gradiflow in the purification or separation of biomolecule from a biological contaminant.

In a fourth aspect, the present invention consists in biomolecule substantially free from biological contaminants purified by the method according to the first aspect of the present invention.

In a fifth aspect, the present invention consists in use of biomolecule according to the third aspect of the present invention in medical and veterinary applications.

In a sixth aspect, the present invention consists in a substantially isolated biomolecule substantially free from biological contaminants.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In order that the present invention may be more clearly understood a preferred forms will be described with reference to the accompanying drawings.

### BRIEF DESCRIPTION OF DRAWINGS

FIG. 1. Shows a 4 to 20% native electrophoresis gel of samples and a Western blot of samples. Lanes 1 and 2 of both the electrophoresis gel and the Western blot show stream 1 at 0 minutes (human plasma with bovine brain homogenate) and at 300 minutes (albumin depleted human plasma) respectively. Lanes 3 through 8 show stream 2 and 0 minutes, 60 minutes (albumin), 120 minutes, 180 minutes, 240 minutes, and 300 minutes, respectively.

FIG. 2. Shows a 4 to 20% native electrophoresis gel of samples and a Western blot of samples. Lanes 1, 2, and 3 of both the electrophoresis gel and Western blot show stream 1 and 0 minutes (human plasma with bovine brain homogenate), at 240 minutes (albumin depleted human plasma), and at 300 minutes (IgG depleted human plasma) respectively. Lanes 4 through 9 show stream 2 at 0 minutes, 60 minutes (IgG), 120 minutes, 180 minutes, 240 minutes, and 300 minutes, respectively.

FIG. 3. Samples from up and downstream were taken at time intervals (x-axis) during the isolation of albumin from plasma. Albumin was measured in the samples by mixing with BCG reagent and reading the absorbance of 630 nm. The concentration of albumin in each sample was calculated from the standard curve, and multiplied by the volume of the up-or downstream to obtain the Total HSA in the up- and downstream (y-axis). All samples were assayed for prion using a sandwich ELISA, and recording the absorbance values at 450 nm (second y-axis).

FIG. 4. Samples from the second phase of an IgG separation were taken from both up- and downstreams (U/S and D/S respectively) at 30 minute intervals. The samples were assayed for endotoxin using a LAL Chromogenic assay (Cape Cod Assoc.)

FIG. 5. HSA was purified from endotoxin spiked plasma. Samples were taken from up- and downstream at 30 minute intervals during a 90 minute purification (x-axis). Analysis of the samples using a LAL Chromogenic assay was performed to establish the endotoxin concentration (y-axis) in the samples.

FIG. 6. Four to 25% native gel electrophoresis of samples from an HSA purification from endotoxin spiked plasma. Lane 1 contains molecular weight markers. Lane 2 contains starting plasma sample, Lanes 3-5 contain upstream samples at time 30, 60, and 90 minutes. Lanes 6-9 contain downstream samples at time 0, 30, 60 and 90 minutes, respectively.

### MODES FOR CARRYING OUT THE INVENTION

### EXAMPLE I

Virus Removal During Plasma Protein Purification

Contamination with virus is a major concern when purifying plasma proteins, such as IgG and human serum albumin (HSA). A contaminant virus can potentially infect a patient receiving the contaminated plasma products. A virus that infects bacteria is known as a phage, and they are readily detected by examining culture plates for cleared zones in a coating or lawn of bacteria.

1. IgG Purification Procedure

IgG is the most abundant of the immunoglobulins, representing almost 7000 of the total immunoglobulins in human serum. This class of immunoglobulins has a molecular mass of approximately 150 kDa and consists of 4 subunits, two of which are light chains and two of which are heavy chains. The concentration of IgG in normal serum is approximately 10mg/mL.

IgGs are conventionally purified using Protein A affinity columns in combination with DEAB-cellulose or DEAE-Sephadex columns. The main biological contaminants in IgG isolations are β-lipoprotein and transferrin. The product of conventional protein purification protocols is concentrated using ultrafiltration. Immunoaffinity can also be used to isolate specific IgGs.

Platelet free plasma was diluted one part in three with Tris-borate, pH 9.0 running buffer and placed in stream 1 56 of separation apparatus 200 and spiked with either Lambda or T7 phage to a concentration of approximately 10⁸ pfu/mL (plaque forming units/mL). A potential of 250V was placed across a separating membrane 34 with a molecular weight cut off of 200 kDa and with 3 kDa restriction membranes 30 and 32. A membrane 34 of this size restricts IgG migration while allowing smaller molecular weight contaminants to pass through the membrane 34, leaving IgG and other large molecular weight compounds in the stream 1 56. A second purification phase was carried out using a GABA/Acetic acid buffer, pH 4,6 with a 500 kDa cut off separating membrane 34 and with 3 kDa restriction membranes 30 and 32. A potential of 250V reversed polarity was placed across the system resulting in IgG migration through the separation membrane 34 leaving other high molecular weight contaminants stream 1 56.

Examination of samples taken at 30 minutes intervals was made on reduced SDS-PAGE 4-20% gels.

One hundred and fifty microliter samples were taken at each time point sample and mixed with 100 iL of appropriate Escherichia coli culture (Strain HB101 was used for T7 and strain JM101 for Lambda). The mixtures were incubated for 15 minutes at 37° C. and then added to 2.5 mL of freshly prepared molten soft agar, and vortexed. The mixtures were poured over culture plates of Luria Agar, and incubated at 37° C. overnight. The plates were inspected for the presence of virus colonies (plaques) in the lawn of E. coli and the number of plaques was recorded. If the virus had infected the entire E. coli population, the result was recorded as confluent lysis.

2. HSA Purification Procedure

Albumin is the most abundant protein component (50 mg/mL) in human plasma and functions to maintain blood volume and oncotic pressure. Albumin regulates the transport of protein, fatty acids, hormones and drugs in the body. Clinical uses for HAS purification include blood volume replacement during surgery, shock, serious burns and other medical emergencies. Albumin is 67 kDa and has an isoelectric point of approximately 4.9. The protein consists of a single subunit and is globular in shape. About 440 metric tons of albumin is used annually internationally with worldwide sales of US $1.5 billion.

Albumin is currently purified using Cohn fractionation and commercial product contains many contaminants in addition to multimers of albumin. The high concentration, globular nature and solubility of albumin make it an ideal candidate for purification from plasma using the separation technology of the present invention.

Pooled normal plasma was diluted one in three with Tris-Borate (TB) 10 running buffer, pH 9.0 and spiked with approximately 10⁸pfu/mL of Lambda or T7 phage. The mixture was placed in stream 1 56 of the separation apparatus 200. Albumin was isolated from platelet free plasma in a one-phase process using the charge of albumin at a pH above its isoelectric point (pI) and its molecular weight. Thus a cartridge 100 having a 75 kDa cutoff separation membrane 34 and two 50 kDa restriction membranes 30 and 32 was used. The albumin was removed from high molecular weight contaminants by its migration through the separation membrane 34 while small molecular weight contaminants dissipated through the 50 kDa restriction membrane 30. Samples were taken at regular intervals throughout a 90 minute run.

The presence of the purified HSA in the stream 2 was demonstrated by examination by SDS-PAGE. Virus was detected as previously described in the IgG purification procedure.

3. Fibrinogen Purification Procedure

Commercially, fibrinogen has a role as fibrin glue, which is used to arrest bleeding and assist in the wound healing process. Fibrinogen is an elongated molecule of 340 kDa that consists of three non-identical subunit pairs that are linked by a disulfide knot in a coiled coil conformation. The isoelectric point of fibrinogen is 5.5 and it is sparingly soluble as compared to other plasma proteins.

Fibrinogen is conventionally purified from plasma by a series of techniques including ethanol precipitation, affinity columns and traditional electrophoresis. This process takes about 48-72 hours and the harsh physical and chemical stresses placed on fibrinogen are believed to denature the molecule. Cryo-precipitation is the first step in the production of Factor VIII and involves the loss of most of the fibrinogen in plasma. Processing of this waste fibrinogen is of considerable interest to major plasma processors and provides an opportunity to demonstrate the rapid purification of fibrinogen from cryo-precipitate using the separation technology of the present invention.

Cryo-precipitate 1, produced by thawing frozen plasma at 4° C. overnight was removed from plasma by centrifugation at 10000 g at 4° C. for 5 minutes. The precipitate was re-dissolved in Tris-Borate buffer (pH 9.0) and placed in stream 1 56 of separation apparatus 200. Stream 1 56 was spiked with either Lambda or T7 phage to a concentration of approximately 10⁸pfu/mL. A potential of 250V was applied across a cartridge 100 having a 300 kDa separation membrane 34 for a period of 2 hours. Stream 2 66 was replaced with fresh buffer 38 at 30 minute intervals. A second cartridge 100 was then inserted having a 500 kDa cutoff separation membrane 34. A second phase was used to concentrate the fibrinogen through the second cartridge 100 at pH 9.0. Stream 2 66 was harvested at 60 minutes. The product was dialyzed against PBS pH 7.2 and analyzed for clotting activity by the addition of calcium and thrombin (final concentrations 10 mM and 10 NIG unit/mL respectively).

The presence of purified fibrinogen was confirmed by examination on reduced SDS PAGE 4-20% gels. The presence of either T7 or Lambda in the time point samples was tested using the previously described method.

4. Results of IgG, HSA and Fibrinogen Purification

The procedures described successfully purified IgG, albumin and fibrinogen as judged by electrophoresis. Neither T7 nor Lambda phage were detected in the stream 2 products, but were present in the stream 1 samples.

### Example II

Prion diseases have recently become a focus of intense research, especially in Europe and the US. The unique mechanism of replication and transmission, and the ability of related prion diseases to transmit between species have contributed significantly to this area. While there is no epidemiological evidence yet to support Creutzfeldt-Jakob disease (CJD) transmission by human blood or blood products, a related disease in transmissible spongiform encephalopathy (TSE). Animal studies have highlighted that whole blood and its components such as plasma and buffy boat, are capable of transmitting the disease. The emergence of a new variant CJD has raised increased concerns about the safety of blood components and plasma products derived from vCJD-infected donors. Recent risk-minimization strategies have included a ban on the use of UK-sourced plasma for the preparation of licensed blood products and leukodepletion of blood donations. Although processes such as precipitation, depth filtration and chromatographic procedures during plasma fractionation, have the potential to remove TSE agents to the limit of detection, whether or not these processes would have been capable of completely removing all the spiked TSE infectivity is uncertain. Using normal bovine prion protein as a surrogate for the abnormal form associated with TSE infectivity, complete prion clearance of the input spike was achieved during the purification of human albumin, immunoglobulin and α1-proteinase inhibitor from human plasma by the present invention.

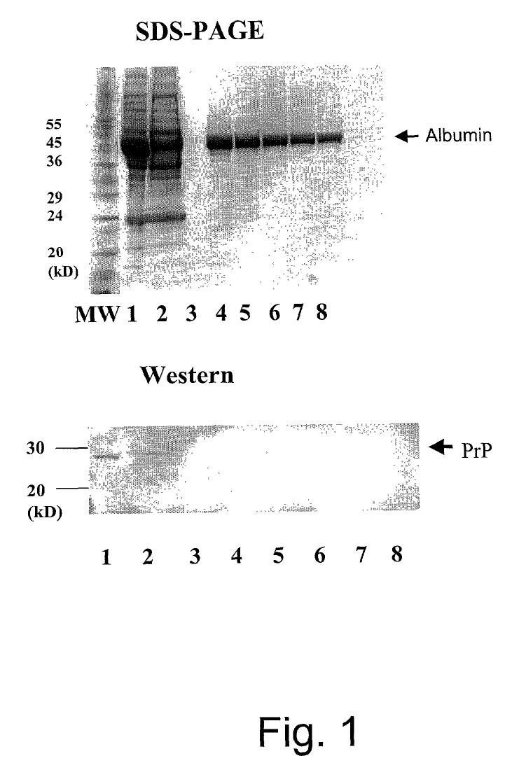

Human plasma (⅓ ratio), were mixed with bovine brain homogenate, containing PrP^{C} and placed in stream 1 of a separation apparatus. Purification of albumin was performed at 250 V using a cartridge with a separation membrane of 150 kDa and two restriction membranes of 5 kDa and 20 mM Tris-Borate (TB) running buffer, pH 9.0. Stream 2 fractions were collected every 60 minutes over a 5-hour run. The running conditions were selected such that the running buffer pH was higher than albumin pI, but lower than pI of PrP^{C} and the separation of albumin and PrP^{c} was achieved based on their charge differences. The presence of purified albumin in stream 2 was examined by SDS-PAGE and the yield was measured using a Bromocresol Green Assay (Trace Scientific). Anti-PrP Western blot, used to detect PrP^{C}, showed that PrP^{C} remained in stream 1 and stream 2 albumin fractions were completely free of PrP^{C} as shown in FIG. 1.

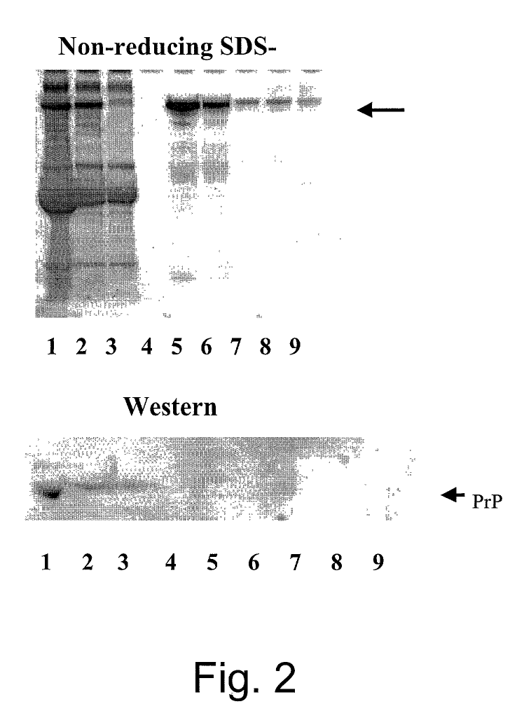

Similar partitioning experiments were carried out in the purification of Immunoglobulin from human plasma. Human plasma (⅓ ratio) were mixed with bovine brain homogenate, containing PrP^{C} and placed in stream 1 of a separation apparatus. By using an 800 kDa separation membrane, 5 kDa and 80 kda-restriction membranes cartridge and 30 mM GABA/Acetic Acid (pH 4.6), the spiked bovine PrP^{c} was completely removed from stream 2 fractions which contained the purified human Immunoglobulin as shown in FIG. 2. The separation of IgG and PrP^{c} was achieved based on their size differences.

1. Albumin Quantitation

Fifty microliters of sample from each time point were diluted with 50 uL of PBS buffer 36, 38. A 20 uL aliquot of each diluted sample was placed in a microplate well. A standard curve with a maximum concentration of 40 mg/mL albumin was prepared using PBS as the diluent. The standard curve dilutions were also placed in the microplate (2T1 plasma/well). The bromocresol green reagent was added to all the wells (200 uL/well) and the absorbance at 630 nm was read using a Versamax microplate reader. A standard curve was drawn on a linear scale and the concentration of albumin in stream 1 56 and stream 2 66 samples were read from the curve. The volume in the appropriate stream 1 56 or stream 2 66 at the time of sampling was multiplied by the concentration of each sample, thus providing a value for the total HSA present in each stream.

2. Prion Detection

Anti-PrP Western Blot

After subjecting the samples to SDS-PAGE analysis, a semi-dry transfer of proteins onto the nitrocellulose (NC) was performed for 1 hour at 15V. The NC was then blocked at 37° C. for 30 minutes before being incubated with anti-PrP antibody, R029, (Prionics, Switzerland) and subsequently incubated with HRP-conjugated secondary antibody. Western blot was developed by Enhanced Chemiluminescence ECL™ (Amersham Pharmacia Biotech).

3. Results

Albumin was transferred to the stream 2 66 and was detected in the BCG assay (FIG. 1), and visualized on a 4-20% SDS polyacrylamide electrophoresis gel. By Western blot analysis PrP^{C} was detected in stream 1 56 and no prion was detected in the stream 2 samples.

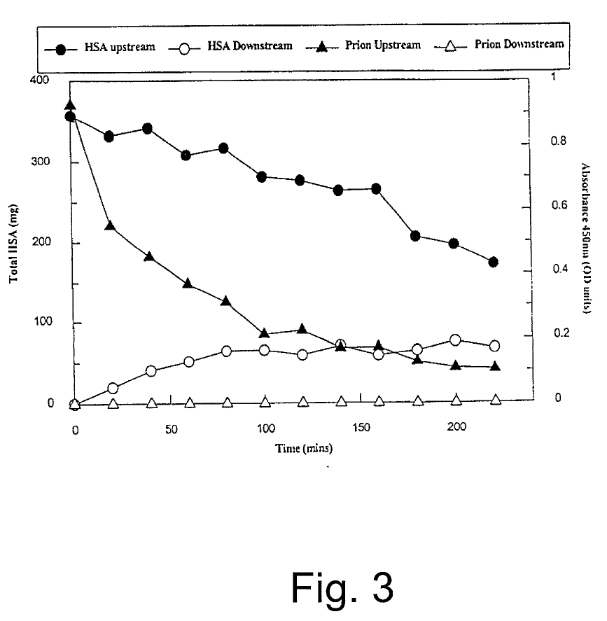

Referring to FIG. 3, samples from stream 1 and stream 2 were taken at time intervals (x-axis) during the isolation of albumin from plasma. Albumin was measured in the samples by mixing with BCG reagent and reading the absorbance of 630 nm. The concentration of albumin in each sample was calculated from the standard curve, and multiplied by the volume of stream 1 or stream 2 to obtain the total HSA in stream 1 or stream 2 (y-axis).

### EXAMPLE III

Endotoxin Removal During Plasma Protein Purification

Contamination with bacterial endotoxin is a major concern when purifying plasma proteins, such as IgG and HSA. Endotoxins are a lipopolysaccharide derived from the lipid membrane of gram negative bacteria. The presence of endotoxin in a human blood fraction therapeutic can lead to death of the receiving patients.

1. IgG Purification Procedure

Platelet free plasma was diluted one part in three with Tris-borate, pH 9.0 running buffer and placed in stream 1 67 of a separation apparatus 200 and spiked with purified E. coli endotoxin to a concentration of 600 EU/mL (endotoxin units/mL). A potential of 250V was placed across a cartridge 100 having a separating membrane 34 with a molecular weight cut off 30 of 75 kDa restriction membranes 30 and 32 with a molecular weight cut off of 50 kDa. A separation membrane 34 of this size restricts IgG migration whilst allowing smaller molecular weight contaminants to pass through the membrane 34, leaving IgG and other large molecular weight compounds in the stream 1 56. A second purification phase was carried out using a MES/bis-tris buffer, pH 5.4 with a cartridge 100 having a separating membrane 34 with a molecular weight cut off of 500 kDa restriction membranes 30 and 32 with a molecular weight cut off of 80 kDa. A potential of 250V reversed polarity was placed across the system resulting in IgG migration through the separation membrane 34 leaving other high molecular weight contaminants in stream 1 56.

Examination of samples taken at 30 minutes intervals was made on reduced SDS-PAGE 4-20% gels. Endotoxin was tested for using a LAL Pyrochrome Chromogenic assay purchased from Cape Cod Associates. All samples were appropriately diluted and the endotoxin assay was performed according to the manufacturer instructions.

2. HSA Purification Procedure

Pooled normal plasma was diluted one in three with Tris-Borate (TB) running buffer, pH 9.0 and spiked with 600 EU/mL of purified endotoxin. The mixture was placed in stream 1 56 of a separation apparatus 200. Albumin was isolated from platelet free plasma in a one-phase process using the charge of albumin at a pH above its pI and its molecular weight. Thus a cartridge 100 having a separation membrane 34 with 75 kDa cutoff and restriction membranes 30 and 32 with 50 kDa cutoffs. The albumin was removed from high molecular weight contaminants by its migration through the separation membrane 34 while small molecular weight contaminants dissipated through the 50 kDa restriction membrane 30. Samples were taken at regular intervals throughout a 180 minutes run.

The presence of the purified HSA in stream 2 66 was demonstrated by examination by SDS-PAGE. Endotoxin was tested for in both stream 1 56 and stream 2 66 samples using a LAL Chromogenic assay supplied by Cape Cod Associates. All samples were appropriately diluted and the endotoxin assay was performed according to the manufacturer instructions.

3. Results of IgG and HSA Purification

Stream 1 56 and stream 2 66 samples taken at 30 minute intervals during the second phase of an IgG purification from endotoxin spiked plasma were tested for endotoxin using a LAL Chromogenic assay. The results showed that the endotoxin was almost entirely found in the stream 1 at all time points (FIG. 2). Stream 2 66 contained only 0.7% of the initial endotoxin. Reduced SDS-PAGE examination showed that IgG had been successfully isolated in the stream 2.

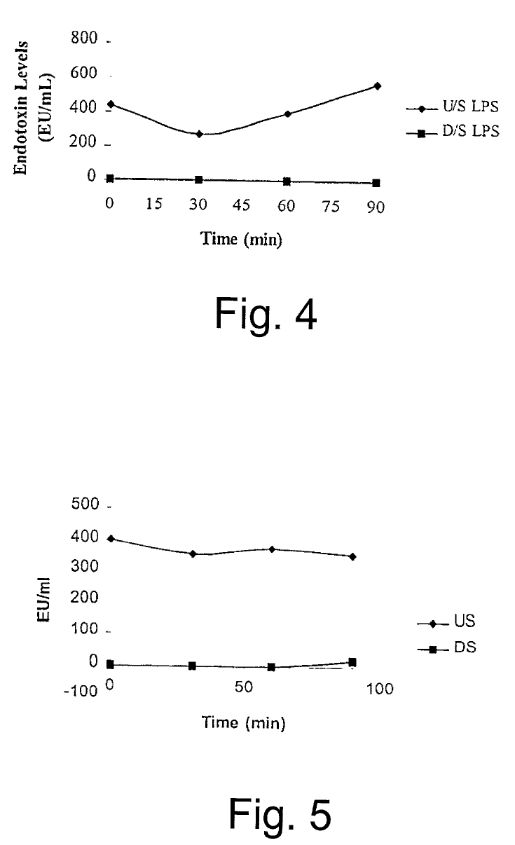

Referring to FIG. 4, samples from the second phase of an IgG separation were taken from both stream 1 56 and stream 2 66 (S1 and S2 respectively) at 30 minute intervals. The samples were assayed for endotoxin using a LAL Chromogenic assay (Cape Cod Assoc.)

Analysis of samples taken at 30 minute intervals during the purification of HSA from plasma spiked with endotoxin found the majority of endotoxin remained in stream 1 56. Only 4% of the total endotoxin was found in the stream 2 66 at the end of the run (FIG. 3). Native PAGE examination confirmed the presence of purified HSA in the stream 2 samples (FIG. 4).

Referring to FIG. 5, HSA was purified from endotoxin spiked plasma. Samples were taken from up- and stream 2 at 30 minute intervals during a 90 minute purification (x-axis). Analysis of the samples using a LAL Chromogenic assay was performed to establish the endotoxin concentration (y-axis) in the samples.

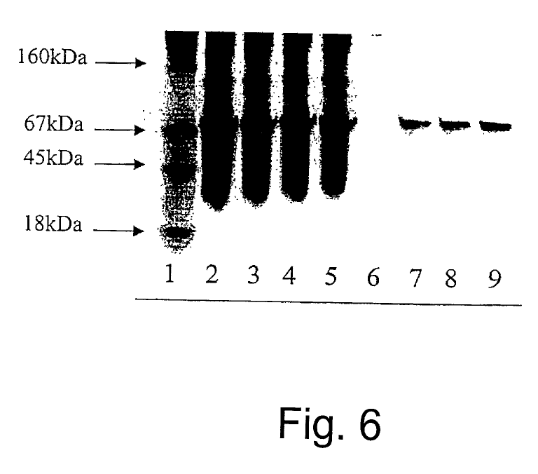

Referring to FIG. 6, 4 to 20% native gel electrophoresis of samples from an HSA purification from endotoxin spiked plasma. Lane 1 contains molecular weight markers, Lane 2 contains starting plasma sample, Lanes 3-5 contain stream 1 samples at time 30, 60, and 90 minutes, Lanes 6-9 contain stream 2 samples at time 0, 30, 60 and 90 minutes, respectively.

### Example IV

Bacteria Removal During Plasma Protein Purification

Contamination with bacteria is a major concern when purifying plasma proteins, such as IgG and HSA. Contaminant bacteria can potentially infect a patient receiving plasma products, or during pasteurization of plasma products when bacteria dies releasing dangerous endotoxins that are harmful to the patient. Bacteria are easily detected by culturing samples on nutrient agar plates.

1. IgG Purification Procedure

Platelet free plasma was diluted one part in three with Tris-borate, pH 9.0 running buffer and placed in stream 1 56 of the separation apparatus 200 and spiked with E. coli to a concentration of 4×10⁸ cells/mL. A potential of 250V was placed across a cartridge 100 having a separation membrane 34 with 200 kDa cutoff and restriction membranes 30 and 32 with 100 kDa cutoffs. A separation membrane 34 of this size restricts IgG migration while allowing smaller molecular weight contaminants to pass through the separation membrane 34, leaving IgG and other large molecular weight compounds in stream 1 56. A second purification phase was carried out using a GABA/Acetic acid buffer, pH 4.6 with a cartridge 100 having a separation membrane 34 with 500 kDa cutoff and restriction membranes 30 and 32 with 3 kDa cutoffs. A potential of 250V reversed polarity was placed across the system resulting in IgG migration through the separation membrane 34 leaving other high molecular weight contaminants in stream 1 56.

Examination of samples taken at 30 minutes intervals was made on reduced SDS-PAGE 4-20% gels. Twenty microliters of stream 1 56 or 100 uL of stream 2 66 samples were spread plated onto Luria agar culture plates. The plates were incubated for 24 hours at 37° C., and the number of colonies was counted.

2. HSA Purification Procedure

Pooled normal plasma was diluted one in three with Tris-Borate (TB) running buffer, pH 9.0 and spiked with approximately 4×10⁸ cells/mL of E. coli. The mixture was placed in stream 1 56 of a separation apparatus 200. Albumin was isolated from platelet free plasma in a one-phase process using the charge of albumin at a pH above its pI and its molecular weight. Thus a cartridge 100 with a 75 kDa cutoff separation membrane 34 and 50 kDa cutoff restriction membranes 30 and 32 was used. The albumin was removed from high molecular weight contaminants by its migration through the separation membrane 34 while small molecular weight contaminants dissipated through the 50 kDa restriction membrane 30. Samples were taken at regular intervals throughout a 90 minutes run.

The presence of the purified HSA in stream 2 was demonstrated by examination by SDSPAGE. Bacteria were detected as previously described above.

4. Results of IgG, and HSA Purification

The procedures described successfully purified IgG, and albumin as judged by electrophoretic examination. The stream 2 samples containing the purified protein products did not contain detectable E. coli colonies, while stream 1 56 samples produced greatly in excess of 500 colonies/plate.

It is possible to purify proteins such as IgG, albumin and fibrinogen from plasma, while simultaneously removing contaminating virus by the methods according to the present invention.

The present invention has shown to be able to separate or retain spiked prior protein from or in plasma, and thus allows simultaneous removal of prior protein during albumin or IgG purification from plasma.

Evidence has been provided by the present inventors that it is possible to purify proteins such as IgG and albumin from plasma, while simultaneously removing endotoxin contamination in the starting plasma using the separation technology of the present invention.

Furthermore, it has been found that it is also possible to purify proteins such as IgG, and albumin from plasma, while simultaneously removing contaminating bacteria.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive. Other features and aspects of this invention will be appreciated by those skilled in the art upon reading and comprehending this disclosure. Such features, aspects, and expected variations and modifications of the reported results and examples are clearly within the scope of the invention where the invention is limited solely by the scope of the following claims.

What is claimed:

1. A method for concurrently isolating at least a portion of both a selected compound and biological contaminants from a fluid stream, the method comprising:
(a) directing a first fluid stream having a selected pH and including at least one biological contaminant and a selected compound so as to flow along a first selective membrane;
(b) directing a second fluid stream along the first selective membrane so as to be isolated from the first fluid stream thereby;
(c) directing a third fluid stream separated from one of the first and second fluid streams by a second selective membrane;
(d) applying at least one voltage potential across at least the first and second fluid streams, wherein the application of such at least one voltage potential causes movement of at least a portion of at least one of the selected compound and the biological contaminants though the first selective membrane into the second fluid stream, wherein the second selective membrane has a preselected pore size that allows selective migration of components in at least one of the first and second fluid streams through the second selective membrane into the third fluid stream; and
(e) maintaining step (d) until at least one of the fluid streams contains a desired purity of the selected compound.

2. The method according to claim 1 wherein the first selective membrane has a preselected pore size so as to allow selective migration of components in the first fluid stream through the first selective membrane into the second fluid stream and selectively retain other components in the first fluid stream.

3. The method according to claim 1 wherein the step of directing the third fluid stream comprises directing the third fluid stream so as to be separated from the second fluid stream by the second selective membrane.

4. The method according to claim 3 wherein the second selective membrane has a preselected pore size so as to substantially prevent at least one of the selected compound and selected biological contaminants removed to the second fluid stream from migrating through the second selective membrane into the third fluid stream and substantially retain the at least one of the selected compound and selected biological contaminants in the second fluid stream.

5. The method according to claim 4 wherein the application of a voltage potential across the third fluid stream causes movement of at least a portion of at least one of the selected compound and selected biological contaminants removed to the second fluid stream through the second selective membrane into the third fluid stream.

6. The method according to claim 4 wherein the method further comprises directing a fourth fluid stream separated from the first fluid stream by a third selective membrane, wherein the preselected pore size of the third selective membrane allows selective migration of components in the first fluid stream through the third selective membrane into the fourth fluid stream.

7. The method according to claim 6 wherein the third selective membrane has a preselected pore size so as to substantially prevent at least one of the any selected compound remaining in the first fluid stream, any biological contaminants remaining in the first fluid stream, and any other compounds remaining in the first fluid stream from migrating through the third selective membrane into the fourth fluid stream and substantially retain the at least one of the selected compound, biological contaminants, and other components in the second fluid stream.

8. The method according to claim 6 wherein the application of a voltage potential across the fourth fluid stream causes migration of at least a portion of at least one of any selected compound remaining in the first fluid stream, any biological contaminants remaining in the first fluid stream, and any other compounds remaining in the first fluid stream through the third selective membrane into fourth fluid stream.

9. The method according to claim 1 wherein the step of directing a third fluid stream directing the third fluid stream so as to be separated from the first fluid stream by the second selective membrane.

10. The method according to claim 9 wherein the second selective membrane has a preselected pore size so as to substantially prevent at least one of the any selected compound remaining in the first fluid stream, any biological contaminants remaining in the first fluid stream, and any other compounds remaining in the first fluid stream from migrating through the second selective membrane into the third fluid stream and substantially retain at least one of the selected compound, biological contaminants, and other components in the first fluid stream.

11. The method according to claim 9 wherein the application of a voltage potential across the third fluid stream causes migration of at least a portion of at least one of any selected compound remaining in the first fluid stream, any biological contaminants remaining in the first fluid stream, and any other compounds remaining in the first fluid stream through the second selective membrane into third fluid stream.

12. The method according to claim 9 wherein the method further comprises directing a fourth fluid stream separated from the second fluid stream by a third selective membrane, wherein the preselected pore size of the third selective membrane allows selective migration of components in the second fluid stream through the third selective membrane into the fourth fluid stream.

13. The method according to claim 12 wherein the third selective membrane has a preselected pore size so as to substantially prevent at least one of the selected compound and selected biological contaminants removed to the second fluid stream from migrating through the third selective membrane into the fourth fluid stream and substantially retain the at least one of the selected compound and selected biological contaminants in the second fluid stream.

14. The method according to claim 12 wherein the application of a voltage potential across the fourth fluid stream causes movement of at least a portion of at least one of the selected compound and selected biological contaminants removed to the second fluid stream through the third selective membrane into the fourth fluid stream.

15. The method according to claim 1 wherein the method further comprises periodically stopping and reversing the voltage potential to cause movement of at least any compounds of the first fluid stream having entered the first selective membrane to move back into the first fluid stream and wherein substantially not causing any of the selected compound and biological contaminants that have entered the second fluid stream to re-enter the first fluid stream.

16. The method according to claim 1 wherein the first fluid stream further includes a compound from which the selected compound is separated, wherein such compound is selected from the group consisting of blood proteins, immunoglobulins, recombinant proteins, and combinations thereof.

17. The method according to claim 1 wherein the biological contaminant is selected from the group consisting of viruses, bacteria, prions, yeast, lipopolysaccharides, toxins, endotoxins, and combinations thereof.

18. The method according to claim 1 wherein the pH of the first fluid stream is selected by selectively adding a buffer having the required pH and the pH is selected at one of a pH lower than the isoelectric point of the compound, a pH about the isoelectric point of the compound, and a pH higher than the isoelectric point of the compound.

19. A method for concurrently isolating at least a portion of both a selected compound and biological contaminants from a fluid stream, the method comprising:
(a) directing a first fluid stream having a selected pH and including at least one biological contaminant and a selected compound so as to flow along a first selective membrane;
(b) directing a second fluid stream along the first selective membrane so as to be isolated from the first fluid stream thereby;
(c) directing a third fluid stream separated from one of the first and second fluid streams by a second selective membrane;
(d) applying at least one voltage potential across at least the first and second fluid streams, wherein the application of such at least one voltage potential causes movement of at least a portion of the biological contaminants though the first selective membrane into the second fluid stream while the selected compound is prevented from entering the second fluid stream, wherein the second selective membrane has a preselected pore size that allows selective migration of components in at least one of the first and second fluid streams through the second selective membrane into the third fluid stream; and
(e) maintaining step (d) until at least one of the fluid streams contains a desired purity of the selected compound.

20. The method according to claim 19 wherein the first selective membrane has a preselected pore size so as to allow selective migration of components in the first fluid stream through the first selective membrane into the second fluid stream and selectively retain other components in the first fluid stream.

21. The method according to claim 19 wherein the step of directing the third fluid stream comprises directing the third fluid stream so as to be separated from the first fluid stream by the second selective membrane.

22. The method according to claim 21 wherein the second selective membrane has a preselected pore size so as to substantially prevent at least one of the selected compound and selected biological contaminants remaining in the first fluid stream from migrating through the second selective membrane into the third fluid stream and substantially retain at least one of the selected compound and selected biological contaminants in the first fluid stream.

23. The method according to claim 21 wherein the application of a voltage potential across the third fluid stream causes movement of at least a portion of at least one of the selected compound and selected biological contaminants remaining in the first fluid stream through the second selective membrane into the third fluid stream.

24. The method according to claim 21 wherein the method further comprises directing a fourth fluid stream separated from the second fluid stream by a third selective membrane, wherein the preselected pore size of the third selective membrane allows selective migration of components in the second fluid stream through the third selective membrane into the fourth fluid stream.

25. The method according to claim 24 wherein the third selective membrane has a preselected pore size so as to substantially prevent at least one of any biological contaminants removed to the second fluid stream and any other compounds in the second fluid stream from migrating through the third selective membrane into the fourth fluid stream and substantially retain the at least one of the selected biological contaminants and other components in the second fluid stream.

26. The method according to claim 24 wherein the application of a voltage potential across the fourth fluid stream causes migration of at least a portion of at least one of any biological contaminants removed to the second fluid stream, and any other compounds in the second fluid stream through the third selective membrane into fourth fluid stream.

27. The method according to claim 19 wherein the step of directing a third fluid stream directing the third fluid stream so as to be separated from the second fluid stream by the second selective membrane.

28. The method according to claim 27 wherein the second selective membrane has a preselected pore size so as to substantially prevent at least one of any biological contaminants removed to the second fluid stream and any other compounds in the second fluid stream from migrating through the second selective membrane into the third fluid stream.

29. The method according to claim 27 wherein the application of a voltage potential across the third fluid stream causes migration of at least a portion of at least one of any biological contaminants removed to the second fluid stream, and any other compounds in the second fluid stream through the second selective membrane into third fluid stream.

30. The method according to claim 27 wherein the method further comprises directing a fourth fluid stream separated from the first fluid stream by a third selective membrane, wherein the preselected pore size of the third selective membrane allows selective migration of components in the first fluid stream through the third selective membrane into the fourth fluid stream.

31. The method according to claim 30 wherein the third selective membrane has a preselected pore size so as to substantially prevent at least one of the selected compound and selected biological contaminants remaining in the first fluid stream from migrating through the third selective membrane into the fourth fluid stream and substantially retain at least one of the selected compound and selected biological contaminants in the first fluid stream.

32. The method according to claim 30 wherein the application of a voltage potential across the fourth fluid stream causes movement of at least a portion of at least one of the selected compound and selected biological contaminants remaining in the first fluid stream through the third selective membrane into the fourth fluid stream.

33. The method according to claim 19 wherein the method further comprises periodically stopping and reversing the voltage potential to cause movement of at least any compounds of the first fluid stream having entered the first selective membrane to move back into the first fluid stream and wherein substantially not causing any of the selected compound and biological contaminants that have entered the second fluid stream to re-enter the first fluid stream.

34. The method according to claim 19 wherein the first fluid stream further includes a compound from which the selected compound is separated, wherein such compound is selected from the group consisting of blood proteins, immunoglobulins, recombinant proteins, and combinations thereof.

35. The method according to claim 19 wherein the biological contaminant is selected from the group consisting of viruses, bacteria, prions, yeast, lipopolysaccharides, toxins, endotoxins, and combinations thereof.

36. The method according to claim 19 wherein the pH of the first fluid stream is selected by selectively adding a buffer having the required pH and the pH is selected at one of a pH lower than the isoelectric point of the compound, a pH about the isoelectric point of the compound, and a pH higher than the isoelectric point of the compound.

37. A method for isolating at least a portion of a selected compound from a fluid stream, the method comprising:
(a) directing a first fluid stream having a selected pH and including at least a selected compound so as to flow along a first selective membrane;
(b) directing a second fluid stream along the first selective membrane so as to be isolated from the first fluid stream thereby;
(c) directing a third fluid stream separated from one of the first and second fluid streams by a second selective membrane;
(d) applying at least one voltage potential across at least the first and second fluid streams, wherein the application of such at least one voltage potential causes movement of at least a portion of the selected compound though the first selective membrane into the second fluid stream, wherein the second selective membrane has a preselected pore size that allows selective migration of components in at least one of the first and second fluid streams through the second selective membrane into the third fluid stream; and
(e) maintaining step (d) until at least one of the fluid streams contains a desired purity of the selected compound.

38. The method according to claim 37 wherein the first selective membrane has a preselected pore size so as to allow selective migration of components in the first fluid stream through the first selective membrane into the second fluid stream and selectively retain other components in the first fluid stream.

39. The method according to claim 37 wherein the method further comprises directing a fourth fluid stream separated from the other of the first and second fluid streams by a third selective membrane, wherein the preselected pore size of the third selective membrane allows selective migration of components in the other of first and second fluid streams through the third selective membrane into the fourth fluid stream.

40. A method for isolating at least a portion of a selected compound from a fluid stream, the method comprising:
(a) directing a first fluid stream having a selected pH and including at least a selected compound so as to flow along a first selective membrane;
(b) directing a second fluid stream along the first selective membrane so as to be isolated from the first fluid stream thereby;
(c) directing a third fluid stream separated from one of the first and second fluid streams by a second selective membrane;
(d) applying at least one voltage potential across at least the first and second fluid streams, wherein the application of such at least one voltage potential causes movement of at least a portion of components in the first fluid stream through the first selective membrane into the second fluid stream while the selected compound is prevented from entering the second fluid stream, wherein the second selective membrane has a preselected pore size that allows selective migration of components in at least one of the first and second fluid streams through the second selective membrane into the third fluid stream; and
(e) maintaining step (d) until at least one of the fluid streams contains a desired purity of the selected compound.

41. The method according to claim 40 wherein the first selective membrane has a preselected pore size so as to allow selective migration of components in the first fluid stream through the first selective membrane into the second fluid stream and selectively retain other components in the first fluid stream.

42. The method according to claim 40 wherein the method further comprises directing a fourth fluid stream separated from the other of the first and second fluid streams by a third selective membrane, wherein the preselected pore size of the third selective membrane allows selective migration of components in the other of first and second fluid streams through the third selective membrane into the fourth fluid stream.

43. A system for concurrently isolating at least a portion of both a selected compound and biological contaminants from a fluid stream, the system comprising:
means for directing a first fluid stream having a selected pH and including at least one biological contaminant and a selected compound so as to flow along a first selective membrane;
means for directing a second fluid stream along the first selective membrane so as to be isolated from the first fluid stream thereby;
means for directing a third fluid stream separated from one of the first and second fluid streams by a second selective membrane; and
means for applying at least one voltage potential across at least the first and second fluid streams, wherein the application of such at least one voltage potential causes movement of at least a portion of at least one of a selected compound and the biological contaminants though the first selective membrane into the second fluid stream, wherein the preselected pore size of the second selective membrane allows selective migration of components in at least one of the first and second fluid streams through the second selective membrane into the third fluid stream.

44. A system for concurrently isolating at least a portion of both a selected compound and biological contaminants from a fluid stream, the system comprising:
means for directing a first fluid stream having a selected pH and including at least one biological contaminant and a selected compound so as to flow along a first selective membrane;
means for directing a second fluid stream along the first selective membrane so as to be isolated from the first fluid stream thereby;
means for directing a third fluid stream separated from one of the first and second fluid streams by a second selective membrane; and
means for applying at least one voltage potential across at least the first and second fluid streams, wherein the application of such at least one voltage potential causes movement of at least a portion of the biological contaminants though the first selective membrane into the second fluid stream while the selected compound is prevented from entering the second fluid stream, wherein the preselected pore size of the second selective membrane allows selective migration of components in at least one of the first and second fluid streams through the second selective membrane into the third fluid stream.
